(19) United States
(12) Patent Application Publication
(10) Pub. No.: US 2007/0225496 A1
(43) Pub. Date: Sep. 27, 2007

(54) PROCESS FOR PREPARING TEMOZOLOMIDE

(76) Inventors: Raghavendracharyulu Venkata Palle, Hyderabad (IN); Anant Madhavrao Marathe, Hyderabad (IN); Amarendhar Manda, Medak (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/690,158

(22) Filed: Mar. 23, 2007

Related U.S. Application Data
(60) Provisional application No. 60/807,626, filed on Jul. 18, 2006.

(30) Foreign Application Priority Data
Mar. 24, 2006 (IN) 538/CHE/2006

(51) Int. Cl. C07D 257/12

(52) U.S. Cl. 544/179

(57) ABSTRACT

The present invention relates to a process for preparing temozolomide.

### INTRODUCTION TO THE INVENTION

The present invention relates to a process for the preparation of temozolomide and intermediates thereof.

Temozolomide is the adopted name for a drug compound having the chemical name 3,4-dihydro-3-methyl-4-oxoimidazo[5,1-d]-as-tetrazine-8-carboxamide, and having structural Formula I.

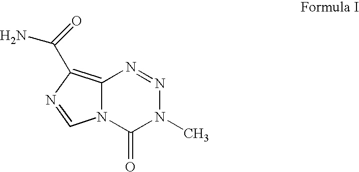

Temozolomide is an imidazotetrazine derivative that exhibits anti-tumor activity and is available in the market under the brand name TEMODAR® in the form of capsules containing 5 mg, 20 mg, 100 mg, or 250 mg of temozolomide.

U.S. Pat. No. 5,260,291 discloses temozolomide and its pharmaceutically acceptable salts. This patent also describes a process for the preparation of temozolomide, comprising condensation of 5-diazo-5H-imidazole-4-carboxylic acid amide with methyl isocyanate to afford temozolomide of Formula I.

U.S. Pat. No. 4,797,419 discloses a process for the preparation of N′-methyl-N,N-diphenyl urea, comprising the reaction of diphenyl carbomyl chloride with methylamine using chloroform in the presence of triethylamine.

U.S. Pat. No. 4,141,913 discloses a process for generating lower alkyl- and cycloalkyl-isocyanates by pyrolyzing an appropriately selected aryl urea, in the absence of solvent.

However, the method described in U.S. Pat. No. 5,260,291 suffers from the fact that during the condensation with methyl isocyanate the reaction mixture is not safe to handle on an industrial or lab scale. The hazards are toxicological, flammability and the explosive nature of the reaction mixture.

The process of the present invention is a convenient process for the preparation of temozolomide with desired purity and yield by using better preparation techniques, which are ecofriendly, cost-effective, robust and well suited for use on an industrial scale.

### SUMMARY OF THE INVENTION

The present invention relates to a process for the preparation of temozolomide of Formula I with high yield and purity.

In one aspect, the present invention provides a process for the preparation of temozolomide of Formula I, comprising pyrolyzing N′-methyl-N,N-diphenyl urea to form a vapor comprising methyl isocyanate, and reacting the methyl isocyanate with 5-diazo-5H-imidazole-4-carboxylic acid amide of Formula II.

In another aspect, the present invention relates to a process for the preparation of N′-methyl-N,N-diphenyl urea of Formula IIIC comprising reacting diphenyl carbomyl chloride of Formula IIIA with methylamine of Formula IIIB in the presence of water.

An embodiment of the invention provides a process for preparing temozolomide, comprising pyrolyzing N′-methyl-N,N-diphenyl urea to form a vapor comprising methyl isocyanate, and reacting formed methyl isocyanate with 5-diazo-5H-imidazole-4-carboxylic acid amide.

Another embodiment of the invention provides a process for preparing temozolomide, comprising pyrolyzing N′-methyl-N,N-diphenyl urea to form a vapor comprising methyl isocyanate, and reacting condensed methyl isocyanate with a solution comprising 5-diazo-5H-imidazole-4-carboxylic acid amide and dimethylsulfoxide, N,N-dimethylformamide, or N,N-dimethylacetamide as a solvent.

A further embodiment of the invention provides a process for preparing N′-methyl-N,N-diphenyl urea, comprising reacting diphenyl carbomyl chloride with methylamine in the presence of water.

The process of the present invention is simple, improved, efficient, industrially feasible, and ecofriendly reproducing the desired compound of Formula I with high yield and purity.

### BRIEF DESCRIPTION OF THE DRAWINGS

FIG. 1 is an X-ray powder diffraction pattern of temozolomide prepared according to Example 5.

FIG. 2 is a differential scanning calorimetry curve of temozolomide prepared according to Example 5.

FIG. 3 is an infrared absorption spectrum of temozolomide prepared according to Example 5.

### DETAILED DESCRIPTION OF THE INVENTION

The present invention relates to a process for the preparation of temozolomide of Formula I with high yield and purity.

In one aspect, the present invention provides a process for the preparation of temozolomide of Formula I, comprising pyrolyzing N′-methyl-N,N-diphenyl urea to form a vapor comprising methyl isocyanate of Formula III, and reacting the methyl isocyanate with 5-diazo-5H-imidazole-4-carboxylic acid amide of Formula II.

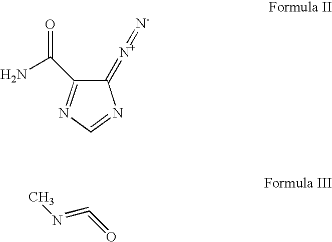

The 5-diazo-5H-imidazole-4-carboxylic acid amide of Formula II of the present invention can be prepared through methods known in the art. For example it can be prepared through the process disclosed in Journal of Organic Chemistry (1961), 26, at page 2396.

The quantity of N′-methyl-N,N-diphenyl urea used to produce methyl isocyanate vapor that is sufficient for complete reaction ranges from about 3 to about 8 times, or about 5 times, the weight of 5-diazo-5H-imidazole-4-carboxylic acid amide of Formula II.

Methyl isocyanate vapor is liberated by the pyrolysis of N′-methyl-N,N-diphenyl urea. The temperatures for the pyrolysis can be range from about 200° C. to about 300° C. The obtained methyl isocyanate vapor can be generated and reacted directly with Formula II in a closed system, without exposure to the hazardous compound by either the production workers or the environment.

The conversion of 5-diazo-5H-imidazole-4-carboxylic acid amide of Formula II to temozolomide of Formula I can be achieved in a single step by condensing the vapor comprising methyl isocyanate in a closed system and directly conducting it into a solution comprising 5-diazo-5H-imidazole-4-carboxylic acid amide and a solvent.

The reaction of methyl isocyanate vapor with 5-diazo-5H-imidazole-4-carboxylic acid amide is carried out until completion. The times required for reaction completion will vary considerably depending on the conditions chosen, frequently being about 1 to about 30 hours, or longer, to form temozolomide of Formula I in desired yields. This reaction frequently proceeds more slowly than a production rate of methyl isocyanate; for example, a particular pyrolysis apparatus might produce a required amount of methyl isocyanate in about 2 hours, but the reaction with 5-diazo-5H-imidazole-4-carboxylic acid amide might require times up to about 24 hours.

The solution of 5-diazo-5H-imidazole-4-carboxylic acid amide is prepared by dissolving 5-diazo-5H-imidazole-4-carboxylic acid amide in a suitable solvent.

Suitable solvents that can be used in the preparation of temozolomide include without limitation thereto: dimethylsulfoxide (DMSO), N,N-dimethylformamide (DMF), N,N-dimethylacetamide (DMA) and the like.

Suitable temperatures for conducting the reaction may range from about 0° C. to about 100° C., or about 25° C. to about 35° C.

After completion of the reaction, the reaction mixture can be diluted with another solvent such as ethyl acetate. Solid can be isolated by precipitation with suitable anti-solvents such as n-hexane, cyclohexane, n-heptane and the like.

The solid can be recovered by any techniques such as filtering, decanting, centrifuging and the like.

Optionally, temozolomide is recrystallized from its solution in a suitable solvent. Suitable solvents that can be used in the recrystallization include but are not limited to acetone and water in ratios from about 1:0.3 to about 1:5, or about 1:3, by volume.

In another aspect, the present invention relates to a process for the preparation of N′-methyl-N,N-diphenyl urea of Formula IIIB, which is useful in the formation of methyl isocyanate of Formula III, comprising reacting diphenyl carbomyl chloride of Formula IIIA with methylamine in the presence of water.

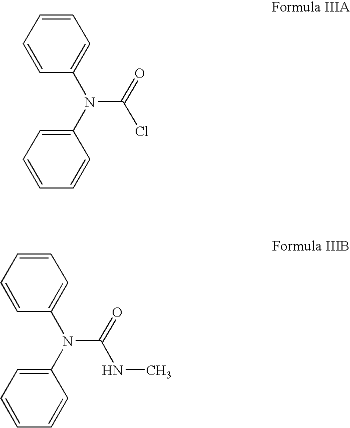

Methylamine used in the preparation of N′-methyl-N,N-diphenyl urea of Formula IIIB can be in the range of about 30 to about 45% in water, or 40% in water, by weight.

Suitable temperatures for the preparation of N′-methyl-N,N-diphenyl urea range from about 0 to about 100° C., or from about 75 to about 80° C.

After completion of the reaction, the reaction mixture is optionally cooled to separate the solid product.

The obtained solid from the process is optionally slurried in water to remove any excess amount of unreacted methylamine.

Temozolomide and its intermediates may optionally be further dried. Drying can be suitably carried out in a tray dryer, vacuum oven, air oven, fluidized bed drier, spin flash dryer, flash dryer and the like. The drying can be carried out at reduced pressures, such as below about 750 mm Hg or below about 50 mm Hg, at temperatures such as about 35° C. to about 120° C. The drying can be carried out for any desired time periods to achieve a desired purity, such as from about 1 to about 26 hours, or longer.

Temozolomide obtained by the above process has been analyzed using high performance liquid chromatography (“HPLC”) with the conditions described in Table 1.

TABLE 1

| Column and | ODS 3 V, 250 × 4.6 mm, 5μ | | | |
|---|---|---|---|---|
| Packing: | | | | |
| Buffer | 5.0 mL of glacial acetic acid in 1000 mL of Milli-Q water, filtered | | | |
| | and degassed. | | | |
| Mobile Phase A: | Buffer solution used as Mobile Phase A | | | |
| Mobile Phase B: | Filtered and degassed methanol as Mobile Phase B | | | |
| | Time | Solution | Solution | |
| | (minutes) | A (% v/v) | B (% v/v) | Elution |
| Gradient: | 0 | 90 | 10 | Isocratic |
| | 8 | 90 | 10 | Isocratic |
| | 25 | 50 | 50 | Linear gradient |
| | 40 | 20 | 80 | Linear gradient |
| | 50 | 10 | 90 | Linear gradient |
| | 55 | 90 | 10 | Re-equilibration |
| | 60 | 90 | 10 | Equilibration |

| | |
|---|---|
| Flow rate: | 1.0 ml/minute |
| Wavelength of | 254 nm |
| detection: | |
| Temperature: | 25 ± 2° C. |
| Injection volume: | 10 μL |
| Sample | 25 mg of temozolomide standard or sample dissolved in diluent |
| preparation | and diluted to 25 mL. Diluted 100 μL of this solution to 100 mL |
| | with diluent. |
| Diluent: | Mix buffer and methanol in a ratio of 90:10 (v/v) |
| Run time: | 60 minutes |

| | ~RT | |
|---|---|---|
| Component | (minutes) | RRT |
| 5-Aminoimidazole-4- | 2.1 | 0.21 |
| carboxamide hydrochloride | | |
| (Formula IV) | | |
| 5-Diazo-5H-imidazole-4- | 6.4 | 0.63 |
| carboxylic acid amide | | |
| (Formula II) | | |
| Temozolomide | 10.2 | 1.00 |
| N′-Methyl-N,N-diphenyl | 35.2 | 3.45 |
| urea (Formula IIIB) | | |
| Diphenyl carbomyl chloride | 42.3 | 4.15 |
| (DPCC) Formula IIIA | | |

Since the retention times (“RT”) can show some variation between repetitive analyses, the relative retention times (“RRT”), calculated by dividing the RT values by the RT value for temozolomide, are used to give more consistent results.

Temozolomide obtained by the process of the present invention typically has a purity not less than about 99%, or about 99.5%, by weight as determined using HPLC.

Temozolomide provided by the process of this invention contains: less than about 0.15% of 5-diazo-5H-imidazole-4-carboxylic acid amide of Formula II; less than about 0.15% of N′-methyl-N,N-diphenyl urea of Formula IIIB; less than about 0.15% of diphenyl carbomyl chloride of Formula IIIA; and less than about 0.15% of 5-aminoimidazole-4-carboxamide hydrochloride of Formula IV.

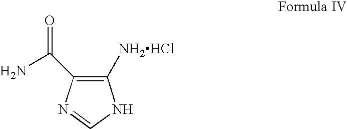

In an embodiment, temozolomide obtained by the process of the present invention has a particle size distribution with: D₁₀ less than about 20 microns; D₅₀ less than about 50 microns; and D₉₀ less than about 100 microns.

The D₁₀, D₅₀ and D₉₀ values are useful ways for indicating a particle size distribution. D₉₀ refers to the value for the particle size for which at least 90 volume percent of the particles have a size smaller than the value given. Likewise D₅₀ and D₁₀ refer to the values for the particle size for which 50 volume percent, and 10 volume percent, respectively, of the particles have a size smaller than the value given. Methods for determining D₁₀, D₅₀ and D₉₀ include laser light diffraction, such as using equipment from Malvern Instruments Ltd. (of Malvern, Worcestershire, United Kingdom). There is no specific lower limit for any of the D values.

In yet another embodiment of the present invention there is provided temozolomide of Formula I having a bulk density of about 0.2 to about 0.5 g/ml and a tapped bulk density of about 0.5 to about 0.8 g/ml. The bulk densities are determined using Test 616 “Bulk Density and Tapped Density,” United States Pharmacopoeia 29, United States Pharmacopeial Convention, Inc., Rockville, Md., 2005, in method 2.

Yet another embodiment of the present invention provides crystalline temozolomdie of Formula I, provided by the above described process, characterized by its X-ray powder diffraction (“XRPD”) pattern and/or its differential scanning calorimetry (“DSC”) curve.

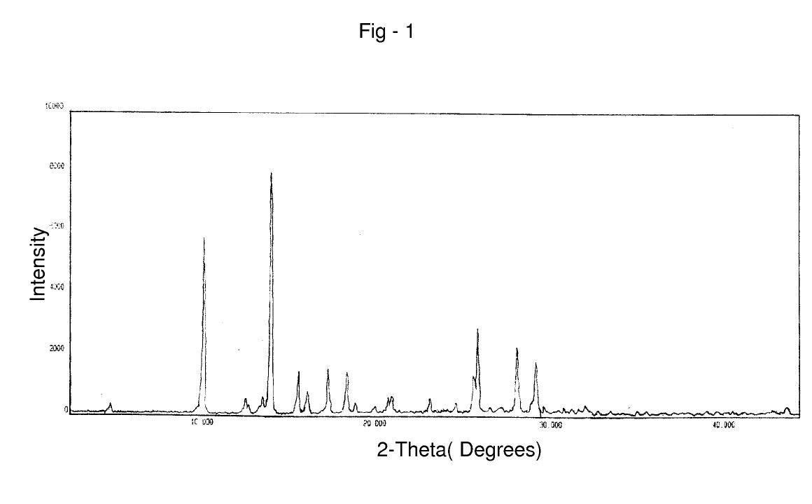

Crystalline temozolomide obtained in the process of present invention is characterized by its XRPD pattern, substantially in accordance with the pattern of FIG. 1. All XRPD data reported herein were obtained using Cu Kα radiation, having the wavelength 1.541 Å and were obtained using a Bruker AXS D8 Advance Powder X-ray Diffractometer.

Crystalline temozolomide is characterized by an XRPD pattern comprising characteristic peaks at about 10.8, 14.8, 16.4, 19.2, 26.6, 28.9, and 30, ±0.2 degrees two theta.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

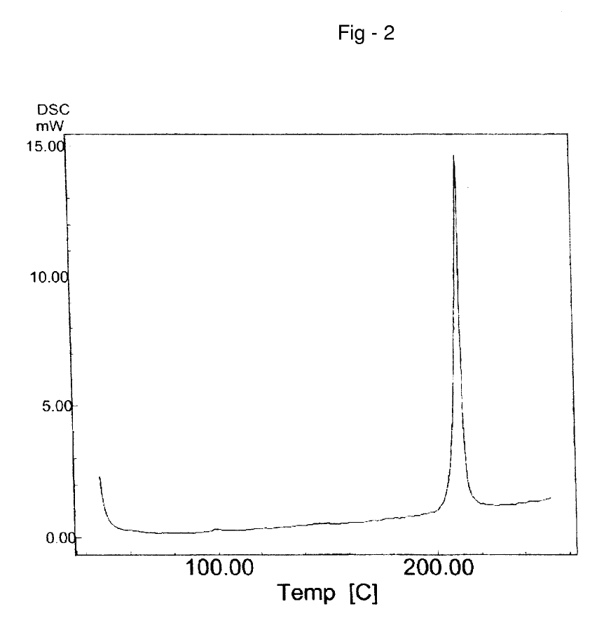

Crystalline temozolomide of the present invention has a characteristic differential scanning calorimetric curve substantially in accordance with FIG. 2, having endothermic peaks at about 211° C. (onset 208° C. and endset 213° C.).

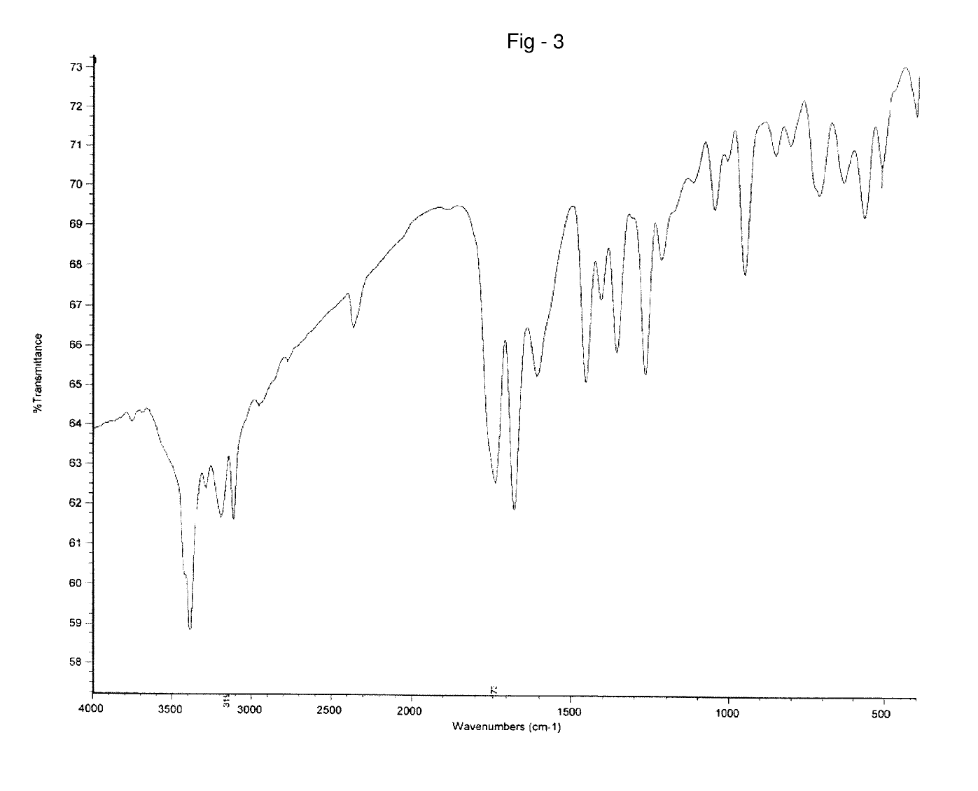

The infrared (IR) absorption spectrum of temozolomide has been recorded on a Perkin Elmer System Spectrum 1 model spectrophotometer, between 450 cm⁻¹ and 4000 cm⁻¹, with a resolution of 4 cm⁻¹ in a potassium bromide pellet, the test compound being at the concentration of 1% by mass. Crystalline temozolomide is characterized by an IR spectrum substantially in accordance with FIG. 3.

Temozolomide obtained using the process of the present invention has a residual solvent content that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

Temozolomide obtained in this invention contains: less than about 500 ppm, or less than about 1000 ppm, of ethyl acetate; less than about 50 ppm, or less than about 100 ppm, of tetrahydrofuran; less than about 1500 ppm, or less than about 2000 ppm, of acetone; less than about 100 ppm, or less than about 500 ppm, of dimethylsulphoxide; and less than about 50 ppm, or less than about 250 ppm, of n-hexane as determined by using gas chromatography (GC).

According to the present invention, essentially all of the methyl isocyanate exposure hazards may be eliminated. The invention may be practiced using N′-methyl-N,N-diphenyl urea, which may be easily and safely handled, transported and stored. Simple pyrolysis of N′-methyl-N,N-diphenyl urea in the specified temperature range yields the desired methyl isocyanate. When methyl isocyanate is reacted with the intermediate 5-diazo-5H-imidazole-4-carboxylic acid amide of Formula III there is directly produced the desired temozolomide product without exposure of the production workers or environment to methyl isocyanate.

The process of the present invention is simple, improved, efficient, industrially feasible, and ecofriendly reproducing the desired compound of Formula I with high yield and purity.

Certain specific aspects and embodiments of the invention will be explained in more detail with reference to the following examples, which are provided by way of illustration only and should not be construed as limiting the scope of the invention in any manner.

### EXAMPLE 1

### Preparation of 5-Diazo-5H-Imidazole-4-Carboxylic Acid Amide (Formula II)

5.64 Kg of sodium nitrite was dissolved in 144 L of demineralized water and then the solution was cooled to 0° C. 12 Kg of 5-aminoimidazole-4-carboxamide hydrochloride was dissolved in hydrochloric acid solution (9.6 L of 36% hydrochloric acid in 86.5 L of water) at 27° C. with stirring for about 15 minutes, then the resultant 5-aminoimidazole-4-carboxamide hydrochloride solution was added slowly portion-wise to the sodium nitrite solution over a period of 60 minutes at 0° C. to get a pink colour. After getting a pink coloured solution, the reaction suspension was stirred for about 10 minutes at about 0° C. The reaction suspension was filtered at 0° C. and the solid suction dried for 15 minutes. The solid obtained was suspended in 120 L of demineralized water and stirred for 30 minutes at 27° C. The reaction suspension was filtered and washed with 24 L of demineralized water, and then the solid was suction dried for 15 minutes. The above step was repeated one more time and then the obtained solid was suspended in 120 L of tetrahydrofuran. The reaction suspension was stirred for 45 minutes at about 27° C. and then the solid obtained was filtered. The solid was washed with 22.5 L of tetrahydrofuran and then the solid was suction dried for 15 minutes. Finally the solid was dried for 6 hours at 45° C. under a vacuum of 580 mm Hg to afford 6.5 Kg of the title compound. Purity: 95.4% by HPLC, loss on drying (LOD): 0.85% w/w.

### EXAMPLE 2

### Preparation of N′-Methyl-N,N-Diphenyl Urea (Formula IIIB)

30 Kg of diphenylcarbamoyl chloride was suspended in 90 L of water followed by stirring for about 5 minutes. 30 L of 40% aqueous monomethylamine solution was charged and the reaction mass was heated to 76.2° C. The obtained reaction solution was stirred for 1 hour at 76.2° C. Thin layer chromatography (“TLC”) was used to determine consumption of diphenylcarbamoyl chloride. After the completion of the reaction, the reaction mixture was cooled to about 27° C. The obtained solid was filtered and then washed with 15 L of demineralized water. The obtained solid was suspended in 60 L of demineralized water and stirred at about 27° C. for 30 minutes. The solid suspension was filtered and the solid was washed with 15 L of demineralized water. The above suspension step was repeated twice to get a suspension pH of about 7.4. Finally the obtained solid was dried for about 20 hours at about 98.1° C. under a vacuum of about 580 mm Hg to afford 28.5 Kg of the title compound.

Purity: 99.8% by HPLC; water by the Karl Fischer (“KF”) method: 0.09% w/w.

### EXAMPLE 3

### Preparation of Temozolomide (Formula I)

3 Kg of N′-methyl-N,N-diphenyl urea was charged into a clean and dry reactor equipped with a condenser and a receiver. 3 L of dimethyl sulfoxide and 600 g of 5-diazo-5H-imidazole-4-carboxylic acid amide were charged into the receiver and then stirred for about 10 minutes at 27° C. The reactor containing N′-methyl-N,N-diphenyl urea was heated to 260° C. for a period of 2 hours, and simultaneously vapours comprising methyl isocyanate were collected into the receiver containing 5-diazo-5H-imidazole-4-carboxylic acid amide, maintained at 30° C. After completion of distillation, the reactor containing N′-methyl-N,N-diphenyl urea was cooled to 80° C. and the condenser was detached. The reaction mass in the receiver was stirred for 30 minutes and then stirred for 24 hours in the dark at 27° C. until the starting material was consumed, as confirmed by TLC. After completion of the reaction, 3 L of ethyl acetate was charged and stirred for about 15 minutes at about 27° C. 3 L of n-hexane was charged and the reaction mixture was stirred at 27° C. for 15 minutes. 3 L of ethyl acetate was charged into the reaction mixture and stirred for 1 hour at 27° C. The separated solid was filtered and then the solid was washed with 3 L of ethyl acetate. The final solid was dried at 60° C. for 24 hours under a vacuum of 580 mm Hg to afford 0.577 Kg of the title compound having a purity by HPLC of 99%.

### EXAMPLE 4

### Purification of Temozolomide (Formula I)

2 Kg of temozolomide was suspended in 4 L of acetone and stirred at 27° C. for 30 minutes. The suspension was cooled to 0° C. and then 4 L of purified water was charged. The obtained mixture was stirred for 30 minutes at 0° C. The suspension was filtered and the solid was suspended in 4 L of acetone. The obtained suspension was cooled to 0° C. and stirred for about 1 hour at 0° C. The suspension was filtered and the obtained solid was suspended in 4 L of acetone and stirred for about 1 hour at 25-30° C., and filtered. The reactor was rinsed with 1 L of acetone and then the wet solid was washed with rinse acetone. The obtained solid was dried at about 60° C. under vacuum to afford 1.52 Kg of the title compound.

### Purity: 99.8% w/w by HPLC.

### Other impurities by HPLC:

5-Aminoimidazole-4-carboxamide of Formula IV: 0.04%.

Diphenyl carbomyl chloride of Formula IIIA: not detected.

N′-methyl-N,N-diphenyl urea of Formula IIIC: not detected.

5-diazo-5H-imidazole-4-carboxylic acid amide of Formula II: 0.04%.

Unidentified impurities: 0.05%.

### Residual solvents content by gas chromatography (GC):

| Solvent | Content (ppm) |
|---|---|
| Acetone | 861 |
| n-Hexane | not detected |
| Ethyl acetate | 449 ppm |
| Tetrahydrofuran | not detected |
| Dimethylsulfoxide | 70 ppm |

### LOD: 0.33% w/w; Moisture content by KF: 0.07% w/w.

Particle size: D₉₀ 47.8 microns; D₅₀ 12.9 microns; and D₁₀: 3.3 microns.
Bulk density: 0.33 g/ml.
Tapped density: 0.71 g/ml.

### EXAMPLE 5

### Crystallization of Temozolomide with Acetone and Water (3:1)

0.2 g of temozolomide was charged into a clean and dry round bottom flask. 6 ml of a mixture of acetone and water (3:1, respectively, by volume) was charged into the flask and then the reaction mixture was heated to 50° C. for reflux. The obtained clear solution was allowed to cool to 27° C. with simultaneous stirring for 1.5 hours. The reaction mixture was kept in an ice bath for 1.5 hours with stirring and then the reaction suspension was filtered. The obtained solid was dried at 60° C. to afford 0.115 g of the title compound.

### Purity 99.68% w/w by HPLC.

### EXAMPLE 6

### Crystallization of Temozolomide with Acetone and Water (3:1)

3.025 L of a mixture of acetone and water (3:1, respectively, by volume) were charged into a round bottom flask with stirring for 10 minutes. 50 g of temozolomide was charged into the above flask and the suspension was stirred for 10 minutes. The reaction suspension was heated to 50° C. to dissolve the temozolomide. After complete dissolution, the solution was cooled to 45° C. A charcoal slurry prepared by suspending 5 g of charcoal in 125 ml of a mixture of acetone and water (3:1, respectively, by volume) was charged to the solution at 45° C. and stirred for 30 minutes. A celite bed prepared with 200 ml of a mixture of acetone and water (3:1, respectively, by volume) was used to filter the suspension, and the flask was rinsed with 25 ml of a mixture of acetone and water (3:1, respectively, by volume). The celite bed was washed with rinse solvent mixture and the obtained filtrate charged into another flask. 2.5 g of temozolomide seeding material was added to the filtrate and stirred for 15 minutes at 40° C. The suspension was cooled to 25° C. slowly with simultaneous stirring for 30 minutes, then was stirred for 1 hour at 25° C. The suspension was cooled to 0° C. and stirred for 1 hour. The obtained suspension was filtered and the solid washed with 100 ml of acetone, then the wet solid was suspended in 250 ml of acetone and stirred for 30 minutes at 25° C. The suspension was filtered and the solid washed with 100 ml of acetone. The obtained solid was dried at 50° C. to afford 30 g of the title compound.

1. A process for preparing temozolomide, comprising pyrolyzing N′-methyl-N,N-diphenyl urea to form a vapor comprising methyl isocyanate, and reacting formed methyl isocyanate with 5-diazo-5H-imidazole-4-carboxylic acid amide.

2. The process of claim 1, wherein a vapor comprising methyl isocyanate is condensed into a solution comprising 5-diazo-5H-imidazole-4-carboxylic acid amide.

3. The process of claim 1, wherein 5-diazo-5H-imidazole-4-carboxylic acid amide is in a solution comprising dimethylsulfoxide, N,N-dimethylformamide, or N,N-dimethylacetamide as a solvent.

4. The process of claim 1, wherein pyrolyzing occurs at temperatures between about 200° C. to about 300° C.

5. The process of claim 1, wherein a weight of N′-methyl-N,N-diphenyl urea that is pyrolyzed is about 3 to about 8 times the weight of 5-diazo-5H-imidazole-4-carboxylic acid amide.

6. Temozolomide obtained according to the process of claim 1, containing less than about 0.15% by weight of any one or more of:
5-diazo-5H-imidazole-4-carboxylic acid amide;
diphenyl carbomyl chloride;
N′-methyl-N,N-diphenyl urea; and
5-aminoimidazole-4-carboxamide hydrochloride.

7. Temozolomide obtained according to the process of claim 1, having D₁₀ less than about 10 microns; D₅₀ less than about 20 microns; and D₉₀ less than about 100 microns.

8. A process for preparing temozolomide, comprising pyrolyzing N′-methyl-N,N-diphenyl urea to form a vapor comprising methyl isocyanate, and reacting condensed methyl isocyanate with a solution comprising 5-diazo-5H-imidazole-4-carboxylic acid amide and dimethylsulfoxide, N,N-dimethylformamide, or N,N-dimethylacetamide as a solvent.

9. The process of claim 8, wherein pyrolyzing occurs at temperatures between about 200° C. to about 300° C.

10. The process of claim 8, wherein a weight of N′-methyl-N,N-diphenyl urea that is pyrolyzed is about 3 to about 8 times the weight of 5-diazo-5H-imidazole-4-carboxylic acid amide.

11. The process of claim 8, wherein reacting occurs at temperatures about 0° C. to about 100° C.

12. The process of claim 8, wherein reacting occurs at temperatures about 25° C. to about 35° C.

13. A process for preparing N′-methyl-N,N-diphenyl urea, comprising reacting diphenyl carbomyl chloride with methylamine in the presence of water.

14. The process of claim 13, wherein methylamine is provided in the form of an aqueous solution.

15. The process of claim 13, wherein methylamine is provided in an aqueous solution at a concentration about 30 to about 45 percent by weight.

16. The process of claim 13, wherein reacting occurs at temperatures between about 70° C. and about 80° C.
